(19) United States
(12) Patent Application Publication
(10) Pub. No.: US 2011/0207873 A1
(43) Pub. Date: Aug. 25, 2011

(54) PREPARATION OF DI(AMINOACETONITRILE)S

(75) Inventors: Ralph M DiGuilio, Spring, TX (US); Matthew W. Forkner, Spring, TX (US); Cheng-Kuang Li, The Woodlands, TX (US)

(73) Assignee: Huntsman Petrochemical LLC, The Woodlands, TX (US)

(21) Appl. No.: 13/124,670

(22) PCT Filed: Oct. 7, 2009
(86) PCT No.: PCT/US09/59775

§ 371 Date: Apr. 18, 2011

Related U.S. Application Data
(60) Provisional application No. 61/112,325, filed on Nov. 7, 2008.
(60) Provisional application No. 61/157,823, filed on Mar. 5, 2009.

(51) Int. Cl. C08G 65/48; C08L 63/00; C08G 18/38; C08G 18/50; C08L 75/12; C07C 255/61

(52) U.S. Cl. 524/500; 525/50; 525/533; 528/68; 524/874; 558/455; 558/430

(57) ABSTRACT

A process for preparing a diaminoacetonitrile which includes reacting by contacting an amine comprising two primary amine groups with a cyanohydrin. The diaminoacetonitrile produced may subsequently be used in the production of polymers and/or as a curing agent for epoxy resins.

### CROSS REFERENCE TO RELATED APPLICATIONS

Not applicable.

### STATEMENT REGARDING FEDERALLY SPONSORED RESEARCH AND DEVELOPMENT

Not applicable.

### FIELD OF THE INVENTION

This disclosure, in general, relates to polyaminoacetonitriles, a process for preparing polyaminoacetonitriles and their use.

### BACKGROUND

It is generally known aminoacetonitriles may be produced by reacting formaldehyde and hydrogen cyanide with a nitrogen source. For example, U.S. Pat. No. 4,478,759 teaches a process for preparing aminoacetonitriles by reacting formaldehyde and hydrogen cyanide with ammonia or alkylamines at a pH below 2. In U.S. Pat. No. 5,008,428, aminoacetonitriles are taught to be produced by contacting in a reactive absorber a gaseous mixture of hydrogen cyanide and ammonia, a gaseous mixture of formaldehyde and unreacted methanol and an additional nitrogen source in the presence of a pH controlled aqueous solution.

EP Pat. No. 0481394 B1 further describes a process in which glycolnitrile is first reacted with an alkylamine to form a reaction product which is subsequently reacted with formaldehyde and hydrogen cyanide so that each hydrogen on the amine nitrogen is replaced by an acetonitrile. U.S. Pat. Nos. 5,817,613, 5,210,271, 2,169,736, and 1,972,465 describe processes for reacting glycolnitrile and monoamines, primarily for further modifying the nitrile group on the aminoacetonitrile to make substituted amino acids or iminodiacetic acid end products. Finally, U.S. Pat. Nos. 3,925,389; 3,067,255; 2,519,803; 2,429,876 and British Pat. No. 798,075 teach processes for reacting glycolnitrile with ethyleneamines as an alternative route to higher ethyleneamines.

As such, there is still a need to find new aminoacetonitrile compounds which are capable as serving as chain extenders and/or curing agents.

### SUMMARY

In one embodiment, a polyaminoacetonitrile is produced by a process which involves reacting by contacting an amine comprising at least two primary amine groups with a cyanohydrin. The reaction may be carried out at a pH above 8, a temperature ranging from about 20° to about 70° C. and atmospheric pressure.

In another embodiment, a process for preparing a polymer includes reacting by contacting a first component comprising at least one isocyanate with a second component comprising a polyaminoacetonitrile produced according to the present invention.

In yet another embodiment, a process for curing a curable composition involves mixing an epoxy resin with a polyaminoacetonitrile produced according to the present invention and applying heat to the curable composition.

In a further embodiment, the present invention provides the polyaminoacetonitriles, polymers and cured products obtained by the processes above.

In a further embodiment, the present invention teaches novel polyaminoacetonitriles.

### DESCRIPTION OF THE PREFERRED EMBODIMENT(S)

The following explanations of terms and methods are provided to better describe the present compounds, compositions and processes, and to guide those of ordinary skill in the art in the practice of the present disclosure. It is also to be understood that the terminology used in the disclosure is for the purpose of describing particular embodiments and examples only and is not intended to be limiting.

In this specification and in the claims which follow, reference will be made to a number of terms which shall be understood to have the following meanings.

The term “alkyl group” refers to a branched or unbranched saturated hydrocarbon group of carbon atoms, such as methyl, ethyl, n-propyl, isopropyl, n-butyl, isobutyl, t-butyl, pentyl, hexyl, heptyl, octyl, decyl, tetradecyl, hexadecyl, and the like.

The term “alkenyl group” refers to a branched or unbranched hydrocarbon group of carbon atoms containing at least one carbon-carbon double bond.

The term “alkynyl group” refers to a branched or unbranched hydrocarbon group of carbon atoms containing at least one carbon-carbon triple bond.

The terms “halogenated alkyl group” or “haloalkyl group” refer to an alkyl group as defined above with one or more hydrogen atoms present on these groups substituted with a halide.

The term “cycloalkyl group” refers to a non-aromatic carbon-based ring composed of at least three carbon atoms. The term may include species with one or more rings, whether connected by sharing a side or by bridging atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulfur, or phosphorous.

The term “aryl group” refers to any carbon-based aromatic group including, but not limited to, benzene, naphthalene, etc. The term “aromatic” also includes a “heteroaryl group” which is defined as an aromatic group that has at least one heteroatom incorporated within the ring of the aromatic group. Examples of heteroatoms include, but are not limited to, nitrogen, oxygen, sulfur, and phosphorous. The aryl group can be substituted with one or more groups including, but not limited to, alkyl, alkynyl, alkenyl, aryl, halide, nitro, ester, ketone, hydroxy, carboxylic acid, or alkoxy, or the aryl group can be unsubstituted.

The term “aralkyl” refers to an aryl group having an alkyl group, as defined above, attached to the aryl group. An example of an aralkyl group is a benzyl group.

The term “hydroxy group” is represented by the formula —OH. The term “alkoxy group” is represented by the formula —OR⁰, where R⁰ can be an alkyl group, optionally substituted with an alkenyl, alkynyl, aryl, aralkyl, cycloalkyl, halogenated alkyl, or heterocycloalkyl group as described above.

The term “hydroxyalkyl group” refers to an alkyl group that has at least one hydrogen atom substituted with a hydroxyl group. The term “alkoxyalkyl group” is defined as an alkyl group that has at least one hydrogen atom substituted with an alkoxy group described above. Where applicable, the alkyl portion of a hydroxyalkyl group or an alkoxyalkyl group can have aryl, aralkyl, halide, hydroxy, or alkoxy groups.

The term “ester” is represented by the formula —OC(O)R¹, where R¹ can be an alkyl, alkenyl, alkynyl, aryl, aralkyl, cycloalkyl, halogenated alkyl, or heterocycloalkyl group described above.

The term “carboxylic acid” is represented by the formula —C(O)OH.

The term “ketone group” is represented by the formula —C(O)R², where R² is an alkyl, alkenyl, alkynyl, aryl, aralkyl, cycloalkyl, halogenated alkyl, or heterocycloalkyl group described above.

The term “carbonyl group” is represented by the formula C═O.

The term “halide” is defined as F, Cl, Br, or I.

The term “nitro group” is represented by the formula NO₂.

The term “alkanoyloxy group,” as used herein, refers to the group R³—C(═O)—O where R³ is an alkyl group of 1 to 5 carbon atoms.

The term “substituted” means that any one or more hydrogens on the designated atom or group is replaced with a selection from the indicated group, provided that the designated atom's normal valence is not exceeded.

The diaminoacetonitriles of the present invention can be prepared in a one step process which involves reacting by contacting an amine comprising two primary amine groups with a cyanohydrin. It has been surprisingly found that such diaminoacetonitriles generate slower reactivity or curing rates when subsequently used in connection with the production of polyurethane, polyurea and polyurethane-urea polymers and cured epoxy resins. Slower reactivity and curing rates is highly desirable since it enables the formation of molded articles and coatings having higher structural integrity. In addition, slower reaction rates allow for the production of caulk and sealant formulations having sufficient gel times for practical use. Furthermore, increased work time through slower cure rate allows formation of smoother and glossier coatings, which are more aesthetically pleasing. Finally, longer working times also provide a benefit in adhesive and sealant applications where having more time to bring two surfaces into contact is critical to success.

The amine comprising at least two primary amine groups which is reacted with the cyanohydrin is an amine compound of the formula (1)

NH₂—R—NH₂ (1)

wherein R is selected from:
(i) a substituted or unsubstituted C₃₋₂₀ cycloalkyl group;
(ii) a substituted or unsubstituted C₆₋₁₄ aryl group;
(iii) a polyether compound of the formula

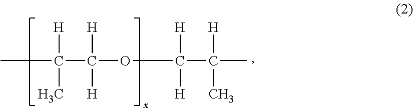

wherein x is from about 2 to about 70 and the molecular weight of the polyether compound of formula (2) is from about 230 g/mol to about 4000 g/mol;
(iv) a polyether compound of the formula (3),

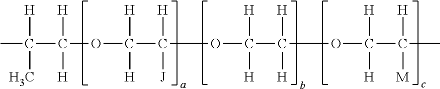

wherein b is from about 2 to about 40, a+c is from about 1 to about 6 and the molecular weight of the polyether compound of the formula (3) is from about 220 g/mol to about 2000 g/mol and wherein J and M are each independently a hydrogen, a methyl group or an ethyl group;
(v) a polyether compound of the formula

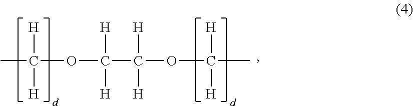

wherein d is 2 or 3;
(vi) a polyether compound of the formula

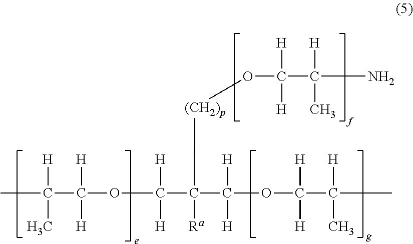

wherein R^{a} is hydrogen or an ethyl group, p is 0 or 1, e+f+g is from about 5 to about 85 and the molecular weight of the polyether compound of the formula (5) is from about 440 g/mol to about 5000 g/mol; and
(vii) a polyoxyalkylene compound of the formula

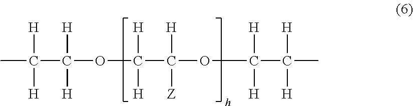

wherein Z is independently selected from hydrogen, a methyl group or an ethyl group, wherein h is an integer, and the compound of formula (6) has a number-average molecular weight ranging from about 100 to about 8000;
(viii) a polyether compound of formula (8):

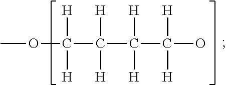

and
(ix) an unsubstituted or substituted C₄₋₁₂ alkyl group; and
(x) a glycol of formula (11):

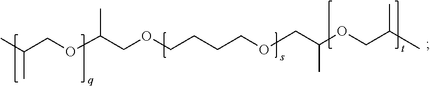

wherein q+t is from about 2 to about 30 and wherein s is from about 5 to about 20; and
(xi) a glycol of formula (12):

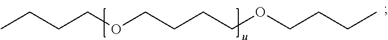

wherein u is from about 1 to about 40.

In one embodiment, the polyether compound of the formula (2) is a compound in which x is from about 2.5 to about 68, preferably from about 6 to about 33. In another embodiment, the polyether compound of the formula (2) is a compound in which x is about 6.1. In still another embodiment, the polyether compound of the formula (2) is a compound in which x is about 33.

In another embodiment, the polyether compound of the formula (3) is selected from a compound in which b is about 2.0 and a+c is about 1.2; b is about 9.0 and a+c is about 3.6; b is about 12.5 and a+c is about 6.0 and b is about 39 and a+c is about 6.0.

In yet another embodiment, the polyether compound of the formula (5) is a compound in which R^{a} is an ethyl group, p is 1 and e+f+g is about 5 to 6. In another embodiment, the polyether compound of the formula (5) is a compound in which R^{a} is hydrogen, p is 0 and e+f+g is 50. In still another embodiment, the polyether compound of the formula (5) is a compound in which R^{a} is hydrogen, p is 0 and e+f+g is 85. In a further embodiment, the polyoxyalkylene compound of the formula (6) is a compound in which Z is hydrogen. In an additional embodiment, the polyoxyalkylene compound of the formula (6) may be a block copolymer compound, a random/block copolymer compound or a random copolymer compound.

In an embodiment, the polypropylene glycol compound of formula (11) has an average s value of about 8, an average q+t value of about 24, and the formula has a molecular weight of about 2000. In another embodiment, the compound of formula (11) has an average s value of about 13.5, an average q+t value of about 17, and the formula has a molecular weight of about 2000. In a further embodiment, the compound of formula (11) has an average s value of about 8, an average q+t value of about 7, and the formula has a molecular weight of about 1000. In yet another embodiment, the compound of formula (11) has an average s value of about 13, an average q+t value of about 7, and the formula has a molecular weight of about 1400.

In an embodiment, the polytetramethylene glycol of formula (12) has a molecular weight of about 232 to about 3000.

Examples of amine compounds of formula (1) include, but are not limited to, phenylenediamine, meta-xylenediamine, bis(aminomethyl)cyclohexylamine, 1,2- and 1,4-diaminocyclohexane, p-aminocyclohexylmethane, and JEFFAMINE® brand polyetheramines, for example, JEFFAMINE® D-4000, D-2000, D-400, D230, HK-511, ED-600, ED-900, ED-2003, EDR-148, EDR-176, T-403, T-3000 and T-5000 polyetheramines (available from Huntsman Corporation). It is also possible to use blends of amine compounds of formula (1). Other derivatives of the compounds above are contemplated, including derivatives with further alkyl or amine substitutions. For example, a compound above with an additional amine substitution would result in a triamine or a tetraamine species for formula (1).

In an embodiment, the amine compound of formula (1) is bis(aminocyclohexyl)methane (PACM) or derivatives thereof. Derivatives of PACM include, without limitation, 2,2′-dimethyl bis(aminocyclohexyl)methane, 3,3′-dimethyl bis(aminocyclohexyl)methane and 3,3′-dimethyl-4,4′-diamino-dicyclohexylmethane (which is sold under the tradename DIMETHYLDICYKAN by BASF Corporation of Mount Olive, N.J.). Other derivatives may include molecules of the structure of PACM, but having further additions or substitutions. Other derivatives may include further amine substitutions of the above structures, which would result in a triamine or in the case of two amine substitutions, a tetraamine.

In another embodiment, the amine compound of formula (1) is an isophorone diamine or a derivative thereof. Derivatives of isophorone diamine include molecules of the structure of isophorone diamine, but having further additions or substitutions. Other derivatives may include further amine substitutions, leading to a triamine or tetraamine.

The amine compound of formula (1) is contacted with a reaction product of a carbonyl containing compound and a cyanide containing compound to produce the polyaminoacetonitrile. The carbonyl containing compound may be an aldehyde, a ketone, or combinations thereof. The cyanide containing compound may be a hydrogen cyanide, an alkali metal cyanide (e.g. NaCN or KCN), or combinations thereof. In one embodiment, the polyaminoacetonitrile may be obtained by reacting a carbonyl containing compound with HCN in the presence of the amine compound of formula (1). In the case of formula (5), a reaction product of the carbonyl containing compound and cyanide containing compound would be with all amine groups on the amine compound of formula (1). Therefore the amine compound of formula (1) including the R group of formula (5) would lead to a triaminoacetonitrile.

In another embodiment, the amine compound of formula (1) is contacted with a cyanohydrin compound of the formula

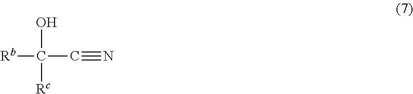

wherein R^{b} and R^{c} are independently selected from hydrogen, substituted or unsubstituted C₁₋₂₀ alkyl, substituted or unsubstituted C₃₋₂₀ cycloalkyl, C₃₋₈ alkenyl, C₃₋₈ alkynyl, and substituted or unsubstituted C₆₋₁₄ aryl. As described below, this compound may be created in-situ by the reaction of a carbonyl compound and hydrogen cyanide.

In one embodiment, R^{b} and R^{c} are independently selected from hydrogen or a C₁₋₂ alkyl. In a further embodiment, R^{b} and R^{c} are hydrogen.

In yet another embodiment, R^{b} or R^{c} is a C₃₋₂₀ cycloalkyl group, preferably a cycloalkyl group having 5 or 6 carbons (i.e. cyclopentyl or cyclohexyl). In another embodiment, R^{b} or R^{c} is a C₃₋₂₀ cycloalkyl which is substituted with one or more C₁₋₄ alkyl, C₁₋₄ alkoxy, hydroxy or C₁₋₄ alkanoyloxy groups.

The cyanohydrin of formula (7) may be formed by methods well known in the art, for example, by reacting a carbonyl containing compound, such as an aldehyde or ketone, with hydrogen cyanide (HCN).

In another embodiment, a cyanohydrin, such as one of formula (7), may be formed in the presence of the amine compound of formula (1), for example, the aldehyde or ketone and excess alkali metal cyanide (e.g. NaCN or KCN) may be reacted in the presence of the amine compound of formula (1) to produce the polyaminoacetonitrile.

In a preferred embodiment, the reaction between the amine comprising at least two primary amine groups and cyanohydrin is carried out by contacting the amine compound of formula (1) with the cyanohydrin compound of formula (7). In one embodiment, the reaction is carried out by admixing the amine compound of formula (1) with the cyanohydrin compound of formula (7) at a mole ratio amine:cyanohydrin of 1:1.0-2.0.

According to another embodiment, the reaction between the amine compound of formula (1) and cyanohydrin compound of formula (7) is carried out at a pH sufficient to allow the cyanohydrin compound to react with the amine compound, for example, at a pH above about 8. In another embodiment, the reaction is carried out at a pH from about 8 to 14. The amine compound of formula (1) is generally sufficiently basic to achieve a pH above 8 in the reaction mixture. Alternatively, the pH may be adjusted prior to or during the reaction using any basic material which does not interfere undesirably with the reaction, such as, sodium hydroxide.

The reaction between the amine compound of formula (1) with the cyanohydrin compound of formula (7) may be carried out batch wise or continuously at a temperature ranging from about 20° C. to about 70° C. The reaction may be conducted under reduced pressure, atmospheric pressure or superatmospheric pressure. Thus, in one embodiment, the reaction is conducted at a temperature range from about 30° C.-to about 40° C. and at atmospheric pressure.

The reaction may also carried out in the presence of water or solvent. Thus, according to one embodiment, the reaction medium comprises the amine compound of formula (1), cyanohydrin compound of formula (7) and water or solvent. The solvent may be one which dissolves both amine and cyanohydrin for example, isopropyl alcohol, or any other aliphatic alcohol with four or fewer carbon atoms. The total amount of water or solvent may range from about 10 percent to about 90 percent, more preferably from about 15 percent to about 50 percent by weight, based on the total amount of amine and cyanohydrin mixture.

According to another embodiment, the polyaminoacetonitriles produced according to the present invention may be used in the production of polymers. As used herein, the term “polymers” includes, but is not limited to, polyureas, polyurethanes, and polyurea-polyurethane hybrids. Thus, in one embodiment, a polymer is produced by a process which involves reacting by contacting a first component comprising at least one isocyanate with a second component comprising at least one polyaminoacetonitrile of the present invention.

As mentioned above, the first component contains at least one isocyanate. The term “isocyanate” includes a wide variety of materials recognized by those skilled in the art as being useful in preparing polyurea, polyurethane and polyurea-polyurethane hybrid polymer materials. Included within this definition are both aliphatic and aromatic isocyanates, as well as one or more prepolymers or quasi-prepolymers prepared using such isocyanates as a starting material, as is generally well known in the art.

Preferred examples of aliphatic isocyanates are of the type described in U.S. Pat. No. 4,748,192, the contents of which are incorporated herein by reference, as well as aliphatic diisocyanates and, more particularly, the trimerized or the biuretic form of an aliphatic diisocyanate, such as hexamethylene diisocyanate (“HDI”), and the bi-functional monomer of the tetraalkyl xylene diisocyanate, such as the tetramethyl xylene diisocyanate. Cyclohexane diisocyanate is also to be considered a useful aliphatic isocyanate. Other useful aliphatic polyisocyanates are described in U.S. Pat. No. 4,705,814, the contents of which are incorporated herein by reference. They include aliphatic diisocyanates, for example, alkylene diisocyanates with 4 to 12 carbon atoms in the alkylene radical, such as 1,12-dodecane diisocyanate, 1,4-tetramethylene diisocyanate, and 1,6-hexamethylene diisocyanate. Also useful are cycloaliphatic diisocyanates, such as 1,3 and 1,4-cyclohexane diisocyanate as well as any mixture of these isomers, 1-isocyanato-3,3,5-trimethyl-5-isocyanatomethylcyclohexane (isophorone diisocyanate); 4,4′-2,2′- and 2,4′-dicyclohexylmethane diisocyanate, H₁₂ MDI (methylene bisphenyl isocyanate), hydrogenated MDI as well as the corresponding isomer mixtures, and the like.

A wide variety of aromatic polyisocyanates may also be used to form a polymer according to the present invention including p-phenylene diisocyanate, polymethylene polyphenylisocyanate, 2,6-toluene diisocyanate, dianisidine diisocyanate, 2,4-toluene diisocyanate, dianisidine diisocyanate, bitolylene diisocyanate, naphthalene-1,4-diisocyanate, bis(4-isocyanatophenyl)methane, bis(3-methyl-3-iso-cyanatophenyl)methane, bis(3-methyl-4-isocyanatophenyl)methane, and 4,4′-diphenylpropane diisocyanate, as well as MDI-based quasi-prepolymers, including without limitation 2,4-methylene bisphenyl isocyanate and 4,4′-methylene bisphenyl isocyanate, such as those available commercially as RUBINATE®9480 RUBINATE® 9484 MDI, and RUBINATE® 9495 MDI (Huntsman Corporation).

Other aromatic polyisocyanates used in the practice of the invention are methylene-bridged polyphenyl polyisocyanate mixtures which have a functionality of from about 2 to about 4. These latter isocyanate compounds are generally produced by the phosgenation of corresponding methylene bridged polyphenyl polyamines, which are conventionally produced by the reaction of formaldehyde and primary aromatic amines, such as aniline, in the presence of hydrochloric acid and/or other acidic catalysts. Known processes for preparing polyamines and corresponding methylene-bridged polyphenyl polyisocyanates therefrom are described in the literature and in many patents, for example, U.S. Pat. Nos. 2,683,730; 2,950,263; 3,012,008; 3,344,162 and 3,362,979, the contents of which are incorporated herein by reference. Usually methylene-bridged polyphenyl polyisocyanate mixtures contain about 20 to about 100 weight percent methylene diphenyl-diisocyanate isomers, with the remainder being polymethylene polyphenyl diisocyanates having higher functionalities and higher molecular weights. Typical of these are polyphenyl polyisocyanate mixtures containing about 20 to about 100 weight percent diphenyl-diisocyanate isomers, of which about 20 to about 95 weight percent thereof is the 4,4′-isomer with the remainder being polymethylene polyphenyl polyisocyanates of higher molecular weight and functionality that have an average functionality of from about 2.1 to about 3.5. These isocyanate mixtures are known, commercially available materials and can be prepared by the process described in U.S. Pat. No. 3,362,979, the contents of which are incorporated herein by reference.

The present invention also includes the use of mixtures of isomers of isocyanates, which are produced simultaneously in a phosgenation reaction, or any blend of two or more isocyanates (including two or more mixtures of isocyanates, or a single isocyanate with a mixture of isocyanates) which are produced using two or more separate phosgenations. One preferred aromatic polyisocyanate is methylene bis(4-phenylisocyanate) or “MDI”. Pure MDI, quasi-prepolymers of MDI, modified pure MDI, etc. are useful to prepare materials according to the invention. Since pure MDI is a solid and, thus, often inconvenient to use, liquid products based on MDI or methylene bis(4-phenylisocyanate) are also useful herein. U.S. Pat. No. 3,394,164 describes a liquid MDI product. More generally, uretonimine modified pure MDI is included also. This product is made by heating pure distilled MDI in the presence of a catalyst. The liquid product is a mixture of pure MDI and modified MDI. The term isocyanate also includes quasi-prepolymers of isocyanates or polyisocyanates with active hydrogen containing materials.

Any of the isocyanates mentioned above may be used as the isocyanate component in the present invention, either alone or in combination with other aforementioned isocyanates. One skilled in the art with the benefit of this disclosure will recognize suitable isocyanates to use for a particular application.

As mentioned above, the second component contains a polyaminoacetonitrile produced according to the present invention. The second component may also contain mixtures of polyaminoacetonitriles produced according to the present invention.

In another embodiment, the first component or second component, or both, may optionally contain at least one polyol. Polyols include, without limitation, polyether polyols; polyester polyols; polycarbonate polyols; acrylic polyols; other polyols such as phenol resin polyols, epoxy polyols, polybutadiene polyols, polyisoprene polyols, polyester-polyether polyols, polymer polyols in which polymers of acrylonitrile or styrene are dispersed or vinyl-addition, urea dispersed polyols, and polyol chain extenders such as 1,4-butane diol catalyst. When a polyol is used, a hybrid polymer is formed such as a polyurea-polyurethane hybrid polymer. This invention teaches the use of polyaminoacetonitriles in such hybrid polymers. One skilled in the art, with the benefit of this disclosure, will recognize other suitable polyols for use in this invention.

In yet another embodiment of the present invention, the first component or second component, or both, may further contain one or more additives. Such additives may include primary polyetheramines; primary amine chain extenders, such as 3-aminomethyl-3,5,5-trimethylcyclohexylamine (also known as IPDA or Isophorone Diamine); aspartic ester amine; diethyl toluene diamine (also known as DETDA, CAS No. 68479-98-1, which is commercially available from the Albemarle Corporation of Baton Rouge, La. under the tradename ETHACURE® 100 curative); dimethylthio toluene diamine (also known as DMTDA, CAS No. 106264-79-3, which is commercially available from the Albemarle Corporation of Baton Rouge, La. under the tradename ETHACURE® 300 curative); secondary amine chain extenders such as N,N-dialkylamino-diphenylmethane (commercially available from Dorf Ketal Chemicals, LLC of Stafford, Tex. under the tradename UNILINK 4200® diamines); Bis(N-sec butylaminocyclohexyl)methane (commercially available from Dorf Ketal Chemicals, LLC of Stafford, Tex. under the tradename CLEARLINK® 1000 diamines); Bis(N-sec butyl 3-methyl aminocyclohexyl)methane (commercially available from Dorf Ketal Chemicals, LLC of Stafford, Tex. under the tradename CLEARLINK® 3000 diamines); N,N′-isopropyl (3-aminomethyl-3,5,5-trimethylcyclohexylamine) (commercially available from Huntsman Petrochemical Corporation under the tradename JEFFLINK® 754 diamines); 1,3 Bis aminomethyl cyclohexane, and its secondary amine byproducts from alkylation with ketones; pigments; anti-oxidant additives; surface active additives; thixotropes; adhesion promoters; UV absorbers; derivatives thereof; and combinations thereof. One skilled in the art, with the benefit of this disclosure, will recognize other suitable additives for use in the polymers and processes of the present invention.

The reaction between the first component and second component to form the polymer occurs by contacting the first component with the second component. To provide a polymer according to the present invention, a first component containing isocyanate is contacted with a second component containing polyaminoacetonitrile, either manually or automatically, using conventional production equipment. During the process for producing polymers, the first and second components are normally kept separated from one another, such as by being contained in separate containers, until being contacted at the time of use. Thus, one embodiment of the present invention provides a system comprising a first vessel containing the first component and a second vessel containing the second component wherein the first component includes at least one isocyanate and the second component includes at least one polyaminoacetonitrile produced according to the present invention. The first vessel or second vessel or both may further contain polyols and other additives described above.

The first component and second component can be contacted by any number of ways known to those skilled in the art such as by blending, mixing, high-pressure impingement mix spraying, low pressure static-mix spray, low pressure static mix dispensing (caulk gun), hand techniques (including mixing by hand or hand tools and then applying the mixture manually with a brush, rollers, or other means), and combinations thereof. One skilled in the art, with the benefit of this disclosure will recognize suitable methods of contacting the first and second components.

Polymers produced according to methods of the present invention are suitable for a wide range of end uses, including without limitation, the following: coatings for concrete, coatings over geotextile, spray on coatings, bridges, bridge pylons, bridge decks, water-proofing layers, tunnels, manholes, fish ponds, secondary containment, skid resistant layers, flooring, garages, aircraft hangars, sewer rehabilitation, water pipes, concrete pipes; coatings for metals, including masking layer for etching process, corrosion protection, ship hulls, ship decks, aircraft carrier decks, submarines, other military vehicles, helicopter rotor blades, bridges, structural members, playgrounds, automotive, truck-bed liners, under-carriage, outer body, rail-road cars and hoppers, trailers, flat bed trucks, 18 wheelers, large dirt moving equipment, rollers, aerospace, tank coatings (inside and out), pipe coating (inside and out); coatings for other substrates such as fiberglass boats, pavement marking, concrete marking, decorative/protective layer over various substrates for movie sets, amusement parks, parade floats, paint-ball props, electronics encapsulation, roofing topcoat for various substrates; coatings for polystyrene, wax, ice, or other media used in prototyping; manufacture of molded articles, such as reaction injection molded and products made using other molding techniques, prototype parts, shoe components, golf balls, decorative parts, automotive parts, bumpers, hubcaps; polyurea foam for sound insulation; thermal insulation; shock absorption; and other end use applications where polyurethane foam is known to be useful in the various arts; caulks for concrete floors and other architectural applications in which a sealant is employed, adhesives for bonding two components in a wide variety of substrates and applications where adhesives are normally employed; and sealants for a wide variety of non-architectural applications, such as on board of sea-going vessels. One skilled in the art, with the benefit of this application will recognize other appropriate uses for embodiments of this invention.

The polyaminoacetonitriles of the present invention may be also used in the curing of epoxy resins. Therefore, another embodiment relates to a curable composition comprising an epoxy resin having, on average, more than one 1,2-epoxy group per molecule, and a polyaminoacetonitrile of the present invention.

For preparation of such compositions according to the invention, the epoxy resins customary in epoxy resin technology are suitable for use. Examples of epoxy resins, having, on average more than 1,2-epoxy group per molecule include: A) polyglycidyl and poly(β-methylglycidyl) esters, obtainable by reacting a compound having at least two carboxyl groups in the molecule with epichlorohydrin and β-methyl-epichlorohydrin, respectively. The reaction is advantageously carried out in the presence of bases.

Aliphatic polycarboxylic acids may be used as the compound having at least two carboxyl groups in the molecule. Examples of such polycarboxylic acids are oxalic acid, succinic acid, glutaric acid, adipic acid, pimelic acid, suberic acid, azelaic acid and dimerised or trimerised linoleic acid. However, cycloaliphatic polycarboxylic acids may also be used, for example tetrahydro-phthalic acid, 4-methyltetrahydrophthalic acid, hexahydrophthalic acid or 4-methylhexahydro-phthalic acid. Aromatic polycarboxylic acids may also be used, for example phthalic acid, isophthalic acid and terephthalic acid.

B) Polyglycidyl or poly(β-methylglycidyl)ethers, obtainable by reacting a compound having at least two free alcoholic hydroxy groups and/or phenolic hydroxy groups with epichlorohydrin or β-methylepichlorohydrin under alkaline conditions, or in the presence of an acid catalyst and subsequently treating with an alkali.

The glycidyl ethers of this kind are derived, for example, from acyclic alcohols, e.g. ethylene glycol, diethylene glycol and higher poly(oxyethylene) glycols, propane-1,2-diol or poly(oxypropylene) glycols, propane-1,3-diol, butane-1,4-diol, poly(oxytetramethylene) glycols, pentane-1,5-diol, hexane-1,6-diol, hexane-2,4,6-triol, glycerol, 1,1,1-trimethylol-propane, pentaerythritol, sorbitol and also from polyepichlorohydrins. Further glycidyl ethers of this kind are derived from cycloaliphatic alcohols, e.g. 1,4-cyclo-hexanedimethanol, bis(4-hydroxycyclohexyl)methane or 2,2-bis(4-hydroxycyclohexyl)-propane, or from alcohols that contain aromatic groups and/or further functional groups, e.g. N,N-bis(2-hydroxyethyl)aniline or p,p′-bis(2-hydroxyethylamino)diphenylmethane. The glycidyl ethers can also be based on mononuclear phenols, such as resorcinol or hydroquinone, or on polynuclear phenols, such as bis(4-hydroxyphenyl)methane, 4,4′-dihydroxybiphenyl, bis(4-hydroxyphenyl)sulfone, 1,1,2,2-tetrakis(4-hydroxyphenyl)ethane, 2,2-bis(4-hydroxyphenyl)propane or 2,2-bis(3,5-dibromo-4-hydroxyphenyl)propane. Further hydroxy compounds that are suitable for the preparation of glycidyl ethers are novolaks, obtainable by condensing aldehydes, e.g. formaldehyde, acetaldehyde, chloral or furfuraldehyde, with phenols or bisphenols that are unsubstituted or substituted by chlorine atoms or by C₁₋₉ alkyl groups, e.g. phenol, 4-chlorophenol, 2-methylphenol or 4-tert-butylphenol.

C) Poly(N-glycidyl) compounds, obtainable by dehydrochlorination of the reaction products of epichlorohydrin with amines containing at least two amine hydrogen atoms. Such amines are, for example, aniline, n-butylamine, bis(4-aminophenyl)methane, m-xylylenediamine or bis(4-methylaminophenyl)methane.

The poly(N-glycidyl) compounds also include, however, triglycidyl isocyanurate, N,N′-diglycidyl derivatives of cycloalkylene ureas, e.g. ethylene urea or 1,3-propylene urea, and diglycidyl derivatives of hydantoins, e.g. 5,5-dimethylhydantoin.

D) Poly(S-glycidyl) compounds, such as di-S-glycidyl derivatives derived from dithiols, e.g. ethane-1,2-dithiol or bis(4-mercaptomethylphenyl)ether.

E) Cycloaliphatic epoxy resins, e.g. bis(2,3-epoxycyclopentyl)ether, 2,3-epoxycyclo-pentylglycidyl ether, 1,2-bis(2,3-epoxycyclopentyloxy)ethane or 3,4-epoxycyclohexylmethyl 3 epoxycyclohexanecarboxylate.

It is also possible, however, to use epoxy resins wherein the 1,2-epoxy groups are bound to different hetero atoms or functional groups; such compounds include, for example, the N,N,O-triglycidyl derivative of 4-aminophenol, the glycidyl ether-glycidyl ester of salicylic acid, N-glycidyl-N′-(2-glycidyloxypropyl)-5,5-dimethylhydantoin and 2-glycidyloxy-1,3-bis(5,5-dimethyl-1-glycidylhydantoin-3-yl)propane.

For preparation of the epoxy resin compositions according to the invention, preference is given to the use of a liquid or solid polyglycidyl ether or ester, especially liquid or solid diglycidyl ether of bisphenol A or bisphenol F or mixtures thereof; or solid or liquid diglycidyl ester of a cycloaliphatic or aromatic dicarboxylic acid; or aliphatic epoxy resins such as trimethylolpropane triglycidyl ethers; or cycloaliphatic epoxy resins, such as hexahydrophthalic acid diglycidyl ester. Mixtures of epoxy resins can also be used.

The polyaminoacetonitriles produced in accordance with the invention can advantageously be used in combination with other epoxy hardeners, especially customary amine hardeners.

Examples of customary amine hardeners include aliphatic, cycloaliphatic, aromatic and heterocyclic amines, for example bis(4-aminophenyl)methane, aniline-formaldehyde resins, benzylamine, n-octylamine, propane-1,3-diamine, 2,2-dimethyl-1,3-propanediamine (neopentanediamine), hexamethylenediamine, diethylenetriamine, bis(3-aminopropyl)amine, N,N-bis(3-amino-propyl)methylamine, triethylenetetramine, tetraethylenepentamine, pentaethylenehexamine, 2,2,4-trimethylhexane-1,6-diamine, m-xylylenediamine, 1,2- and 1,4-diaminocyclohexane, bis(4-aminocyclohexyl)methane, bis(4-amino-3-methylcyclohexyl)methane, 2,2-bis(4-aminocyclohexyl)propane and 3-aminomethyl-3,5,5-trimethylcyclohexylamine (isophorone-diamine), polyaminoimidazolines and polyaminoamides, for example those derived from aliphatic polyamines and dimerised or trimerised fatty acids, polyoxyalkyleneamines, 1,14-diamino-4,11-dioxatetradecane, dipropylene-triamine, 2-methyl-1,5-pentanediamine, N,N′-dicyclohexyl-1,6-hexanediamine, N,N′-dimethyl-1,3-diaminopropane, N,N-diethyl-1,3-diaminopropane, N,N-dimethyl-1,3-diaminopropane, secondary polyoxypropylene-di- and -triamines, 2,5-diamino-2,5-dimethylhexane, bis(amino-methyl)tricyclopentadiene, m-aminobenzylamine, 1,8-diamino-p-menthane, bis(4-amino-3,5-dimethylcyclohexyl)methane, 1,3-bis(aminomethyl)cyclohexane, dipentylamine, bis(4-amino-3,5-diethylphenyl)methane, 3,5-diethyltoluene-2,4-diamine and 3,5-diethyltoluene-2,6-diamine.

Furthermore, the curable epoxy resin/daminoacetonitrile mixtures may comprise tougheners, for example core/shell polymers or the elastomers or elastomer-containing graft polymers known to the person skilled in the art as rubber tougheners. Suitable tougheners are described, for example, in EP-A-449 776, the contents of which are incorporated herein by reference.

In addition, the curable epoxy resin/polyaminoacetonitrile mixtures may comprise fillers, for example metal powder, wood flour, glass powder, glass beads, semi-metal and metal oxides, e.g. SiO₂ (Aerosils, quartz, quartz powder, fused silica powder), corundum and titanium oxide, semi-metal and metal nitrides, e.g. silicon nitride, boron nitride and aluminium nitride, semi-metal and metal carbides (SiC), metal carbonates (dolomite, chalk, CaCO₃), metal sulfates (barytes, gypsum), ground minerals and natural or synthetic minerals chiefly of the silicate series, e.g. zeolites (especially molecular sieves), talcum, mica, kaolin, wollastonite, bentonite and others.

In addition to the additives mentioned above, the curable epoxy resin/polyaminoacetonitrile mixtures may also comprise customary additives, e.g. antioxidants, light stabilisers, plasticisers, dyes, pigments, thixotropic agents, toughness improvers, antifoams, antistatics, lubricants and mould-release agents.

The curing of the epoxy resin compositions according to the invention to form mouldings, coatings or the like is carried out in a manner customary in epoxy resin technology, for example by applying heat, or as described in “Handbook of Epoxy Resins”, 1967, by H. Lee and K. Neville. Thus, in one embodiment, the epoxy resin and polyaminoacetonitrile are admixed to form the curable composition which is then cured by applying heat to the composition.

The invention relates further to the cross-linked products obtained by curing a curable composition comprising an epoxy resin having, on average, more than one 1,2-epoxy group per molecule, and a polyaminoacetonitrile according to the invention.

The curable compositions according to the invention are suitable for use in a variety of application such as a coating composition, adhesive, bonding composition for composite materials or casting resin for the manufacture of mouldings.

In another embodiment, the present invention discloses a polyaminoacetonitrile of formula (9):

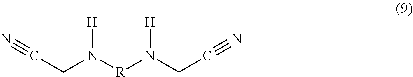

In formula (9), R may be a substituted or unsubstituted C₃₋₂₀ cycloalkyl group or a substituted or unsubstituted C₆₋₁₄ aryl group. R may also be a polyether compound of formula (2), (3), (4), (5), (7), or (8). R may be a polyoxyalkylene compound of the formula (6), a glycol compound of formula (11) or (12) and/or an unsubstituted or substituted C₄₋₁₂ alkyl group.

R may also be a polyether compound of the formula (10):

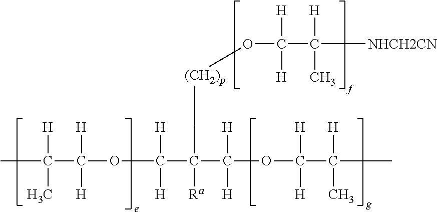

wherein R^{a} is hydrogen or an ethyl group, p is 0 or 1, e+f+g is from about 5 to about 85 and the molecular weight of the polyether compound of the formula (5) is from about 440 g/mol to about 5000 g/mol.

In embodiments of the present invention, the polyaminoacetonitriles of formula (9) may be a diaminoacetonitrile, a triaminoacetonitrile (such as when R is formula (10), or a tetraminoacetonitrile.

The present invention will be further illustrated by a consideration of the following examples, which are intended to be exemplary of the invention. All parts and percentages in the examples are on a weight basis unless otherwise stated.

### EXAMPLES

### Example 1

Synthesis of acetonitrile substituted aminated propoxylated polytetramethylene glycol (1080 mol wt). 150 g aminated propoxylated polytetramethylene (mol wt ˜1000) was combined with 150 g isopropyl alcohol in a 1000 ml flask. 31.4 g glycolnitrile (55% in water) is slowly added keeping the temperature <40 and the reaction mixture digested for 3 hr at room temperature. The mixture is filtered and vacuum stripped at 60° C. to give 142.5 g (88% yield) liquid with an amine value of 1.9 meq/g. The polyaminoacetonitrile produced is represented by:

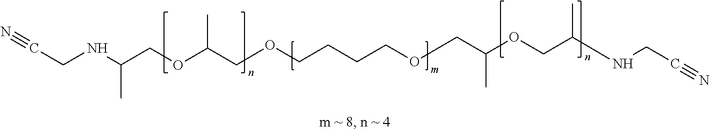

### Example 2

Synthesis of acetonitrile substituted aminated propoxylated polytetramethylene glycol (2480 mol wt). In a similar manner as Example 1, 1000 g aminated propoxylated polytetramethylene (mol wt ˜2400) was converted to give 973 g (94% yield) liquid with an amine value of 0.8 meq/g.

### Example 3

Synthesis of acetonitrile substituted JEFFAMINE® D-2000 polyetheramine. In a similar manner as Example 1,150 g JEFFAMINE® D-2000 polyetheramine was converted to give 146 g (94% yield) liquid with an amine value of 0.96 meq/g. The polyaminoacetonitrile produced is represented by:

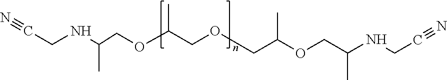

### Example 4

Synthesis of acetonitrile substituted JEFFAMINE® D-2000 polyetheramine (large scale). In a similar manner as Example 1, 24 lb JEFFAMINE® D-2000 polyetheramine was converted to give 23.8 lb (95% yield) liquid with an amine value of 1.0 meq/g.

### Example 5

Synthesis of acetonitrile substituted JEFFAMINE® D-400 polyetheramine. In a similar manner as Example 1,100 g JEFFAMINE® D-400 polyetheramine was converted to give 133.8 g (91% yield) liquid with an amine value of 3.7 meq/g.

### Example 6

Synthesis of acetonitrile substituted JEFFAMINE® D-230 polyetheramine aminated. In a similar manner as Example 1, 100 g JEFFAMINE® D-230 polyetheramine was converted to give 133.8 g (90% yield) liquid with an amine value of 6.5 meq/g.

### Example 7

Synthesis of acetonitrile substituted hexamethylenediamine. In a similar manner as Example 1, 100 g hexamethylenediamine was converted to give 151 g (92% yield) liquid with an amine value of 10.3 meq/g. The polyaminoacetonitrile produced is represented by:

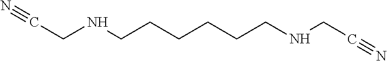

### Example 8

Synthesis of acetonitrile substituted JEFFAMINE® T-403 polyetheramine (a triamine). In a similar manner as Example 1, 150 g JEFFAMINE® T-403 polyetheramine was converted to give 160 g (91% yield) liquid with an amine value of 5.8 meq/g. The polyaminoacetonitrile produced is represented by:

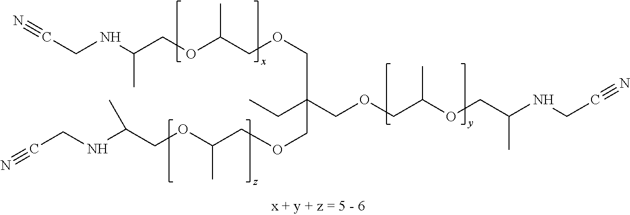

### Example 9

Synthesis of acetonitrile substituted JEFFAMINE® T-3000 polyetheramine (a triamine). In a similar manner as Example 1, 150 g JEFFAMINE® T-3000 polyetheramine was converted to give 147 g (96% yield) liquid with an amine value of 1.0 meq/g.

### Example 10

Synthesis of acetonitrile substituted IPDA. In a similar manner as Example 1, 100 g IPDA was converted to give 134 g (92% yield) liquid with an amine value of 8.1 meq/g. The polyaminoacetonitrile produced is represented by:

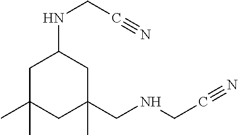

### Example 11

Synthesis of acetonitrile substituted ETHACURE® 100 LC curative: In a similar manner as Example 1, 150 g ETHACURE® 100 LC curative was converted to give 218 g (100% yield) dark liquid with an amine value of 7.8 meq/g.

### Example 12

Synthesis of an acetonitrile substituted IPDA B side composite mixture. 144 g JEFFAMINE® D-2000 polyetheramine, 10 g JEFFAMINE® T-403 polyetheramine, and 74 g IPDA were combined with 40 g isopropyl alcohol in a 500 ml flask. 113.3 g glycolnitrile (55% in water) is slowly added keeping the temperature <40 and the reaction mixture digested for 2 hr at 40° C. The mixture is filtered and vacuum stripped at 70° C. to give 241 g (89% yield) liquid with an amine value of 4.0 meq/g.

### Example 13

Synthesis of an acetonitrile substituted IPDA B side composite mixture (large scale). In a similar manner of Example 12, 12.7 lb JEFFAMINE® D-2000 polyetheramine, 0.88 lb JEFFAMINE® T-403 polyetheramine, and 6.5 lb IPDA were converted to give 20.2 lb (85% yield) liquid with an amine value of 4.0 meq/g.

### Example 14

Synthesis of an acetonitrile substituted ETHACURE® 100 LC curative B side composite mixture. In a similar manner of Example 12, 90 g JEFFAMINE® D-2000 polyetheramine and 90 g ETHACURE® 100 LC curative were converted to give 216 g (85% yield) dark liquid with an amine value of 4.9 meq/g.

### Example 15

Synthesis of dimethylacetonitrile substituted JEFFAMINE® D-400 polyetheramine 100 g JEFFAMINE® D-400 polyetheramine and 250 g isopropyl alcohol were charged to a 1000 ml flask. 43 g hydroxyisobutyronitrile (acetone cyanohydrin) has added keeping the temperature <40° C. The reaction mixture was digested for 3 hr at 45° C. The reaction mixture was filtered and vacuum stripped at 70° C. to give 121 g (91% yield) liquid with an amine value of 3.7 meq/g. The polyaminoacetonitrile produced is represented by:

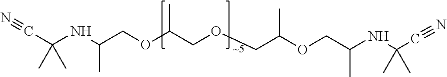

### Example 16

Synthesis of dimethylacetonitrile substituted JEFFAMINE® D-2000 polyetheramine. In a similar manner as Example 15, 300 g JEFFAMINE® D-2000 polyetheramine was converted to give 300 g (94% yield) liquid with an amine value of 0.9 meq/g.

### Example 17

Synthesis of dimethylacetonitrile substituted JEFFAMINE® D-2000 polyetheramine (larger scale). In a similar manner as Example 15, 8000 g JEFFAMINE® D-2000 polyetheramine was converted to give 8326 g (98% yield) liquid with an amine value of 0.9 meq/g.

### Example 18

Synthesis of dimethylacetonitrile substituted JEFFAMINE® D-230 polyetheramine. In a similar manner as Example 15, 100 g JEFFAMINE® D-230 polyetheramine was converted to give 141 g (89% yield) liquid with an amine value of 5.5 meq/g.

### Example 19

Synthesis of dimethylacetonitrile substituted IPDA. In a similar manner as Example 15, 100 g IPDA was converted to give 155 g (87% yield) liquid with an amine value of 6.6 meq/g.

### Example 20

Synthesis of a mixed 50% acetonitrile and 50% dimethylacetonitrile substituted PACM. 80 g PACM and 150 g isopropyl alcohol were charged to a 1000 ml flask. 33.7 g hydroxyisobutyronitrile (acetone cyanohydrin) and 41 g glycolnitrile (55% in water) were combined and added to the PACM solution keeping the temperature <40° C. The reaction mixture was digested for 3 hr at 45° C., filtered, and vacuum stripped at 70° C. to give 95 g (91% yield) sluggish liquid with an amine value of 5.5 meq/g.

### Example 21

Synthesis of mixed 80% acetonitrile and 20% ethylacetonitrile substituted IPDA. 1200 g IPDA and 300 g deionized water are mixed in a 5000 ml flask. 365.7 g HCl (29%) is added in portions and the entire mixture cooled to 10° C. 189.2 g potassium cyanide is added to the flask. 168.8 g propionaldehyde (97%) is mixed with 100 g methanol. The propionaldehyde solution is slowly added to the IPDA mixture maintaining a temperature <15° C. (approx 3 hr). The mixture is allowed to come to room temperature and 1181 g glycolnitrile (55% in water) is slowly added keeping the temperature <40° C. The final mixture is vacuum striped at 70° C. to remove volatile materials and filtered to remove KCl. 1565 g of liquid product was produced (86% yield) with an amine value of 7.7 meq/g. The polyaminoacetonitrile produced is represented by:

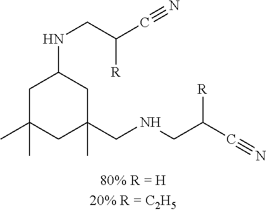

### Example 22

Synthesis of mixed 90% acetonitrile and 10% ethylacetonitrile substituted IPDA. In a similar manner as Example 21, 1200 g IPDA was converted giving 1607 g (90% yield) liquid with an amine value of 7.9 meq/g.

### Example 23

Synthesis of mixed 67% acetonitrile and 33% ethylacetonitrile substituted PACM. In a similar manner as Example 21, 1200 g PACM was converted giving 1430 g (82% yield) liquid with an amine value of 6.5 meq/g. The polyaminoacetonitrile produced is represented by:

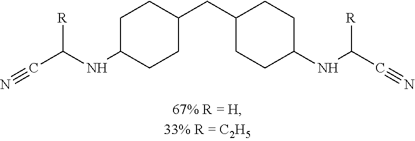

### Example 24

Synthesis of 100% ethylacetonitrile substituted PACM. In a similar manner as Example 21, 100 g PACM was converted giving 150 g (92% yield) friable solid with an amine value of 5.8 meq/g and 65° C. melting point. The polyaminoacetonitrile produced is represented by:

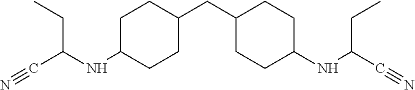

The above-disclosed subject matter is to be considered illustrative, and not restrictive, and the appended claims are intended to cover all such modifications, enhancements, and other embodiments, which fall within the true scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A polyaminoacetonitrile produced by a process comprising:
reacting by contacting an amine compound of the formula
NH₂—R—NH₂ (1)
wherein R is selected from:
(i) a substituted or unsubstituted C₃₋₂₀ cycloalkyl group;
(ii) a substituted or unsubstituted C₆₋₁₄ aryl group;
(iii) a polyether compound of the formula 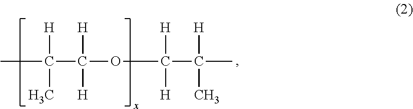
wherein x is from about 2 to about 70 and the molecular weight of the polyether compound of formula (2) is from about 230 g/mol to about 4000 g/mol;
(iv) a polyether compound of the formula (3), 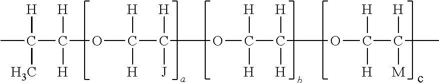
wherein b is from about 2 to about 40, a+c is from about 1 to 6 and the molecular weight of the polyether compound of the formula (3) is from about 220 g/mol to about 2000 g/mol and wherein J and M are each independently a hydrogen, a methyl group or an ethyl group;
(v) a polyether compound of the formula 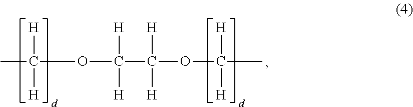
wherein d is 2 or 3;
(vi) a polyether compound of the formula 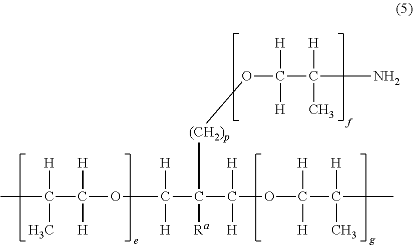
wherein R^{a} is hydrogen or an ethyl group, p is 0 or 1, e+f+g is from about 5 to about 85 and the molecular weight of the polyether compound of the formula (5) is from about 440 g/mol to about 5000 g/mol;
(vii) a polyoxyalkylene compound of the formula 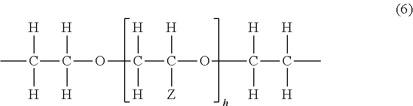
wherein Z is independently selected from hydrogen, a methyl group or an ethyl group, wherein h is an integer and the compound of formula (6) has a number-average molecular weight ranging from about 100 to about 8000;
(viii) a polyether compound of formula (8): 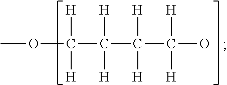
and
(ix) a glycol of formula (11): 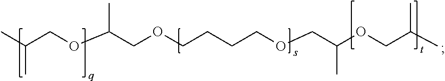
wherein q+t is from about 2 to about 30 and wherein s is from about 5 to about 20; and
(x) a glycol of formula (12): 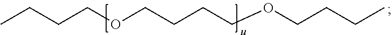
wherein u is from about 1 to about 40;
with a reaction product of a carbonyl containing compound with a cyanide containing compound, or a cyanohydrin compound of the formula 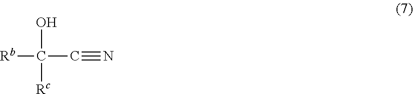
wherein R^{b} and R^{c} are independently selected from hydrogen, substituted or unsubstituted C₁₋₂₀ alkyl, substituted or unsubstituted C₃₋₂₀ cycloalkyl, C₃₋₈ alkenyl, C₃₋₈ alkynyl, and substituted or unsubstituted C₆₋₁₄ aryl.

2. The polyaminoacetonitrile according to claim 1, wherein R^{b} and R^{c} are hydrogen.

3. The polyaminoacetonitrile according to claim 1, wherein R is a polyether compound of the formula (2).

4. The polyaminoacetonitrile according to claim 3, wherein x is about 6.1.

5. The polyaminoacetonitrile according to claim 3, wherein x is about 33.

6. The polyaminoacetonitrile according to claim 1, wherein the amine compound of formula (1) is selected from the group consisting of phenylenediamine, meta-xylenediamine, bis(aminomethyl)cyclohexylamine, 1,2-diaminocyclohexane, 1,4-diaminocyclohexane, and p-aminocyclohexylmethane.

7. The polyaminoacetonitrile according to claim 1, wherein the amine compound of formula (1) is bis(aminocyclohexyl)methane, or a derivative thereof.

8. The polyaminoacetonitrile according to claim 1, wherein the amine compound of formula (1) is an isophorone diamine or a derivative thereof.

9. The polyaminoacetonitrile according to claim 1, wherein R is an amine substituted derivative.

10. The polyaminoacetonitrile according to claim 1, wherein R is a diamine substituted derivative.

11. A process for preparing a polyaminoacetonitrile comprising:
reacting by contacting an amine compound of the formula
NH₂—R—NH₂ (1)
wherein R is selected from:
(i) a substituted or unsubstituted C₃₋₂₀ cycloalkyl group;
(ii) a substituted or unsubstituted C₆₋₁₄ aryl group;
(iii) a polyether compound of the formula 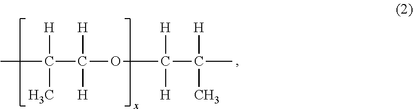
wherein x is from about 2 to about 70 and the molecular weight of the polyether compound of formula (2) is from about 230 g/mol to about 4000 g/mol;
(iv) a polyether compound of the formula (3) 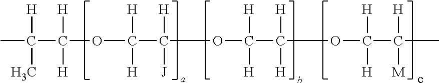
wherein b is from about 2 to about 40, a+c is from about 1 to 6 and the molecular weight of the polyether compound of the formula (3) is from about 220 g/mol to about 2000 g/mol and wherein J and M are each independently a hydrogen, a methyl group or an ethyl group;
(v) a polyether compound of the formula 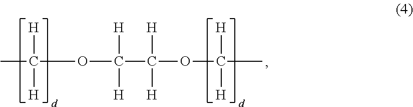
wherein d is 2 or 3;
(vi) a polyether compound of the formula 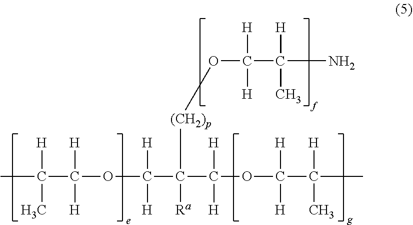
wherein R^{a} is hydrogen or an ethyl group, p is 0 or 1, e+f+g is from about 5 to 85 and the molecular weight of the polyether compound of the formula (5) is from about 440 g/mol to about 5000 g/mol;
(vii) a polyoxyalkylene compound of the formula (6): 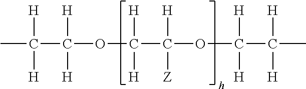
wherein Z is independently selected from hydrogen, a methyl group or an ethyl group, wherein h is an integer and the compound of formula (6) has a number-average molecular weight ranging from about 100 to about 8000; and
(vii) a polyether compound of formula (8): 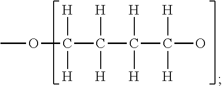
(ix) a glycol of formula (11): 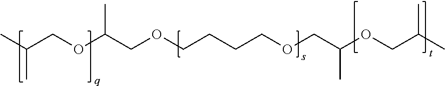
wherein q+t is from about 2 to about 30 and wherein s is from about 5 to about 20; and
(xi) a glycol of formula (12): 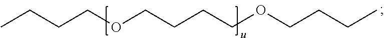
wherein u is from about 1 to about 40;
with a reaction product of a carbonyl containing compound with a cyanide containing compound or a cyanohydrin compound of the formula 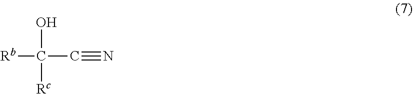
wherein R^{b} and R^{c} are independently selected from hydrogen, substituted or unsubstituted 20 alkyl, unsubstituted or substituted C₃₋₂₀ cycloalkyl, C₃₋₈ alkenyl, C₃₋₈ alkynyl, and substituted or unsubstituted C₆₋₁₄ aryl.

12. The process according to claim 11, wherein the reaction is carried out at a pH of about 8 to 14.

13. The process according to claim 11, wherein the reaction is carried out at a temperature ranging from about 20° to about 70° C. and at atmospheric pressure.

14. The process according to claim 11, wherein the reaction is carried out by admixing the amine compound of formula (1) with the cyanohydrin compound of formula (7) at a mole ratio amine:cyanohydrin of 1:1.0-2.0.

15. A process for preparing a polymer comprising reacting by contacting a first component comprising at least one isocyanate with a second component comprising the polyaminoacetonitrile of claim 1.

16. The process of claim 15, wherein the first component and second component are contacted by blending, mixing, high-pressure impingement mix spraying, low pressure static-mix spray or low pressure static mix dispensing.

17. A polymer produced according to the process of claim 15.

18. A system comprising a first vessel containing a first component and a second vessel containing a second component wherein the first component includes at least one isocyanate and wherein the second component includes at least one diaminoacetonitrile of claim 1.

19. The system according to claim 18, wherein the first vessel or second vessel or both further comprise a polyol.

20. The system according to claim 18, wherein the first vessel or second vessel or both further comprise an additive.

21. A curable composition comprising an epoxy resin having, on average, more than one 1,2-epoxy group per molecule and the diaminoacetonitrile of claim 1.

22. A process for curing a curable composition comprising admixing an epoxy resin having, on average, more than one 1,2-epoxy group per molecule with the diaminoacetonitrile of claim 1 to form the curable composition and applying heat to the curable composition to cure the curable composition.

23. A cross linked product produced according to the process of claim 22.

24. A polyaminoacetonitrile comprising the formula (9) 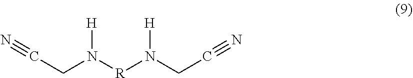
wherein R is selected from:
(i) a substituted or unsubstituted C₃₋₂₀ cycloalkyl group;
(ii) a substituted or unsubstituted C₆₋₁₄ aryl group;
(iii) a polyether compound of the formula 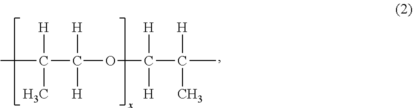
wherein x is from about 2 to about 70 and the molecular weight of the polyether compound of formula (2) is from about 230 g/mol to about 4000 g/mol;
(iv) a polyether compound of the formula (3), 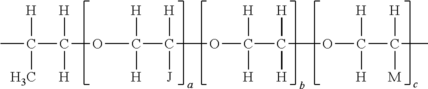
wherein b is from about 2 to about 40, a+c is from about 1 to 6 and the molecular weight of the polyether compound of the formula (3) is from about 220 g/mol to about 2000 g/mol and wherein J and M are each independently a methyl group or an ethyl group;
(v) a polyether compound of the formula 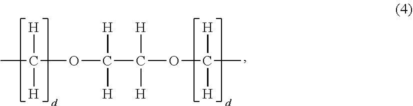
wherein d is 2 or 3;
(vi) a polyether compound of the formula 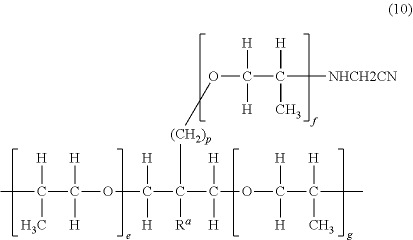
wherein R^{a} is hydrogen or an ethyl group, p is 0 or 1, e+f+g is from about 5 to about 85 and the molecular weight of the polyether compound of the formula (5) is from about 440 g/mol to about 5000 g/mol;
(vii) a polyoxyalkylene compound of the formula (6): 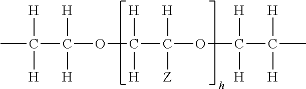
wherein Z is independently selected from hydrogen, a methyl group or an ethyl group, wherein h is an integer and the compound of formula (6) has a number-average molecular weight ranging from about 100 to about 8000; and
(viii) a polyether compound of formula (8): 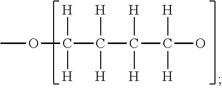
(ix) a glycol of formula (11): 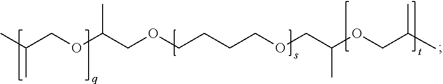
wherein q+t is from about 2 to about 30 and wherein s is from about 5 to about 20; and
(x) a glycol of formula (12): 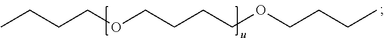
wherein u is from about 1 to about 40.

25. The polyaminoacetonitrile according to claim 1, wherein the amine compound of formula (1) is a triaminoacetonitrile.

26. The polyaminoacetonitrile according to claim 1, wherein the amine compound of formula (1) is a tetraminoacetonitrile.
